(19) United States
(12) Patent Application Publication
(10) Pub. No.: US 2010/0006018 A1
(43) Pub. Date: Jan. 14, 2010

(54) Marine Hull Protective Device and Associated Methods

(76) Inventor: Mark C. Lathem, Boynton Beach, FL (US)

Correspondence Address: JACQUELINE E. HARTT, PH. D.;LOWNDES DROSDICK DOSTER KANTOR & REED, P.A., P.O. BOX 2809, ORLANDO, FL, 32802-2809, US

(21) Appl. No.: 12/501,932

(22) Filed: Jul. 13, 2009

Related U.S. Application Data
(60) Provisional application No. 61/079,832, filed on Jul. 11, 2008.

(51) Int. Cl. B63B 17/00; G01N 33/18

(52) U.S. Cl. 114/361; 73/53.01

(57) ABSTRACT

A device for protecting a hull of a marine vessel includes a cover of water-impermeable material dimensioned to conform to at least a water-contacting portion of a hull of a marine vessel. The cover is removably affixable to the hull, a top edge of the cover extending about the hull in spaced relation above a water line. A drain is positioned so as to lie adjacent a bottom of the hull when the cover is in place on the hull. In use, fresh water is transported into a space between the cover and the hull so as to displace ambient water therefrom. The ambient water can exit the space via the drain, leaving the fresh water within the space as a protective “jacket.” The fresh water serves to prevent galvanic corrosion, pollutant damage, and barnacle encrustation.

### CROSS-REFERENCE TO RELATED APPLICATION

This application claims priority to provisional patent application 61/079,832, filed Jul. 11, 2008.

### BACKGROUND OF THE INVENTION

1. Field of the Invention

The present invention relates to devices and methods for protecting and providing ongoing maintenance for boat hulls, and, more particularly, to such devices and methods for use when the boat remains or is stored in the water.

2. Description of Related Art

The problem of maintaining marine vessel surfaces in the face of corrosive brine and marine fouling agents such as barnacles and pollutants is known to be difficult, time consuming, and expensive. Typically boat hulls have to be repainted every 1-2 years, owing to galvanic corrosion, discoloration, and marine life intrusion, which requires removing the boat from its environment and often transporting the boat to a painting facility.

Any fouling agent on water-contacting surfaces can increase drag and reduce fuel efficiency. Therefore, boat owners have to scrape hulls and other surfaces to remove barnacles and other fouling agents, which is also time consuming and difficult. Therefore, it would be advantageous to provide a device and method for diminishing the need for vessel surface maintenance.

### SUMMARY OF THE INVENTION

The present invention is directed to a device and method for protecting a hull of a marine vessel. The hull-protective device comprises a cover comprising a flexible sheet of water-impermeable material dimensioned to conform to at least a water-contacting portion of a hull of a marine vessel. The cover is removably affixable to the hull, a top edge of the cover extending about the hull in spaced relation above a water line. A drain is positioned so as to lie adjacent a bottom of the hull when the cover is in place on the hull.

In use, fresh water is transported into a space between the cover and the hull so as to displace ambient water therefrom through a drain. Alternatively, the ambient water is first pumped out from the space. The fresh water within the space remains as a protective “jacket,” serving to prevent galvanic corrosion, pollutant damage, and barnacle encrustation on the hull and other water-contacting portions of the boat.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWINGS

FIG. 1 is a side perspective view of an exemplary boat having a hull protective cover of the present invention affixed thereto.

FIG. 2 is a top/side perspective view of a protective cover in position on a boat motor.

FIG. 3 is a top plan view of the motor protective cover prior to sealing.

FIGS. 4A, 4B are top plan views of the protective cover prior to sealing, with FIG. 4A representing the tether embodiment, and FIG. 4B, the flotation element embodiment.

FIG. 5 is a side view of a shore-mounted boom for positioning a protective cover on the hull of a boat.

FIG. 6 is a side view of a boat-mounted boom for positioning the protective cover on the hull of a boat.

FIG. 7 is a flow chart for using the protective cover.

### DETAILED DESCRIPTION OF THE PREFERRED EMBODIMENTS

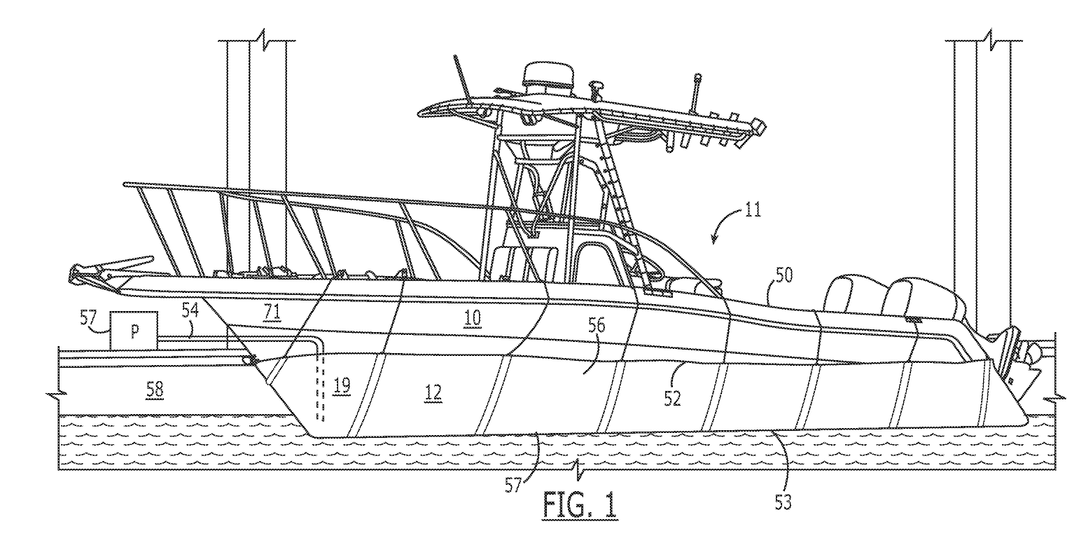

A description of the preferred embodiments of the present invention will now be presented with reference to FIGS. 1-7.

The present invention is directed to a devices and methods for protecting a hull 10 of a marine vessel 11. The hull-protective device 12 in an embodiment (FIGS. 1-4) comprises a cover base 13 comprising flexible sheet 14 of water-impermeable material. The flexibility of the sheet 14 assists in preventing the attachment of barnacles thereto, since such organisms typically require a rigid or semi-rigid surface on which to reside. The sheet 14 is dimensioned to closely conform to at least a water-contacting portion of the hull 10.

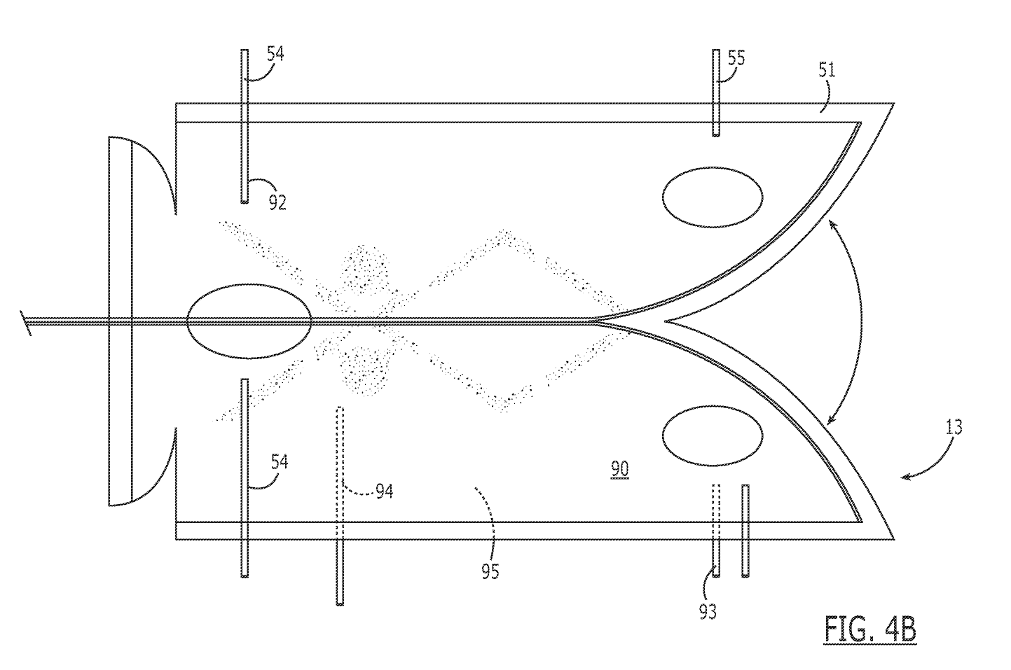

A sheet blank 14 for the cover base (FIG. 4) has substantially parallel side edges 15. A top edge 16 has a curved notch 17 therein, wherein the edges 18 of the notch 17 are sealable to form a boat-hull-conforming fore portion 19. A bottom edge 20 opposed to the top edge 16 has opposed outer portions 21 that are substantially perpendicular to the side edges 15. The bottom edge 20 further has a pair of opposed, arcuate, outwardly extending intermediate portions 22 that join the respective outer portions 21 at an acute angle 23. A central portion 24 of the bottom edge 20 is substantially parallel to the outer portions 21, and joins the respective intermediate portions 22 at an acute angle 25.

A rear aperture 26 is positioned laterally substantially midway between the side edges 15, and vertically adjacent the bottom edge outer portions 21. Two forward apertures 27 can be used to admit other equipment depending from the boat 11.

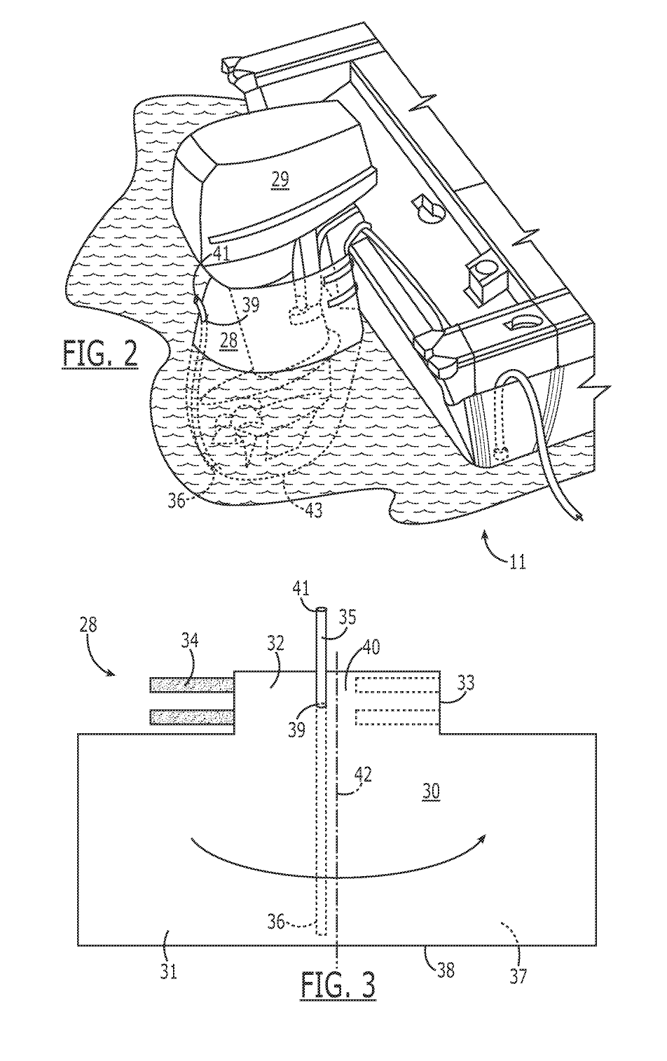

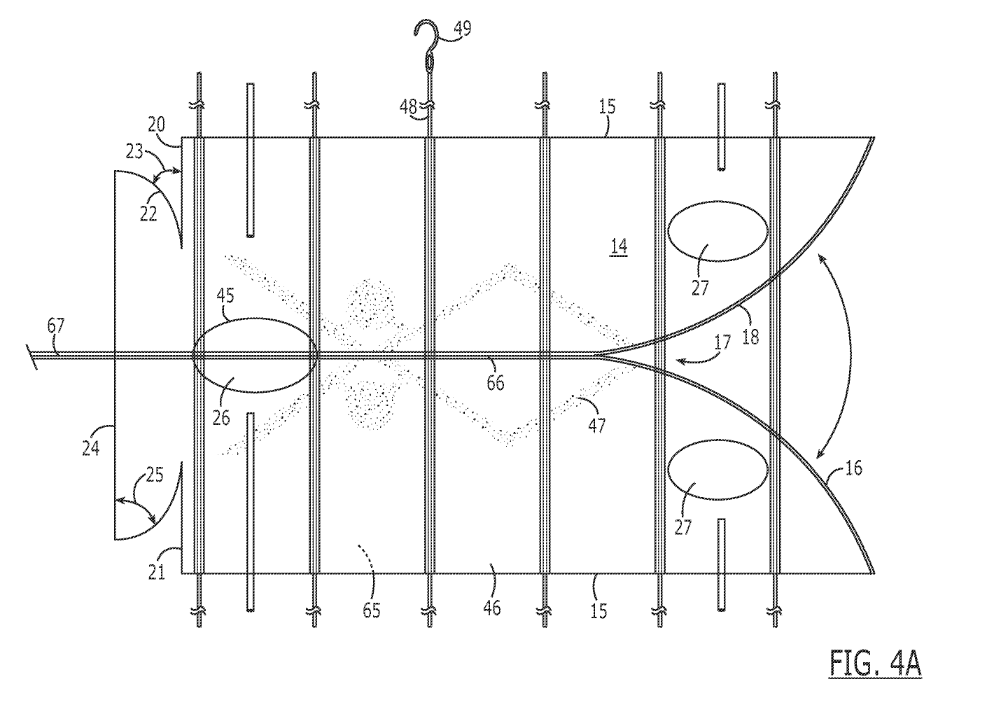

The sheet 14 also can have additional elements such as a pouch 28 (FIGS. 2 and 3) for extending over a motor 29 or other protruding element. The pouch 28 is formed from a unitary flexible sheet 30 having a bottom rectangular portion 31 and an upper, smaller rectangular portion 32. Side edges 33 of the upper rectangular portion 32 have fastening elements 34 extending therefrom, such as, but not intended to be limited to, hook-and-loop-type fasteners. A pouch drain 35 extends from an inner end 36 at an inner surface 37 adjacent a bottom edge 38 of the bottom rectangular portion 31 through an aperture 39 in the upper rectangular portion 32 through to an outer surface 40 thereof, terminating at an outer end 41.

In use, the pouch 28 is formed by folding the sheet 30 along a vertical midline 42 and folding the bottom edge 38 upward to encase the motor 29, as shown in FIG. 2. The pouch drain 35 thus has its inner end 36 adjacent a bottom 43 of the pouch 28 and its outer end 41 extending out from the pouch 28.

Another pouch (not shown) can be joined to the base sheet 14 by attaching an upper edge of that pouch about an edge 45 of the rear aperture 26.

The sheet 14 and pouch sheet 30 can comprise, for example, a plastic, although this is not intended as a limitation. The sheet's inner surface 46 can have affixed thereto, for example, via heat welding, strips 47 of a sponge-like mesh material. these strips 47 provide water circulation and heat exchange channels, and further increase the coefficient of friction between the cover 13 and the hull 10, thereby preventing the cover 11 from being sucked into an intake when water-cooled through-hull equipment is being run.

The cover 13 can be removably affixable to the hull 10 by any means known in the art, for example, via lines, bungee cords, or tethers, although these are not intended as a limitation. In a particular embodiment (FIG. 4A), elastic tethers 48 are affixed to the side edges 15 and the front and back, the tethers 48 having hooks or other affixing means at outer ends 49 thereof, the hooks for attaching the tethers 48 over an upper edge 50 of the boat hull 10.

In another embodiment (FIG. 4B) the cover 13′ can have flotation elements 51, which can be inflatable for ease of storage, affixed thereto, for keeping the upper edges 52 of the cover 13′ above the water line 53.

One or more drain tubes 54,55 are positioned so as to lie with their inner ends 92 adjacent a bottom 57 of the hull 10 when the cover 13,13′ is in place on the hull 10. A first 54 and a second 55 drain tube are illustrated in FIG. 4B.

In an alternate embodiment, the cover 13′ could include a “mirrored” drain system, wherein an intake drain 55 and an outlet drain 54 could have their inner ends 91,92 on an inner surface 90 for typical operation, and further have an intake drain 93 and an outlet drain 94 affixed to an outer surface 95. Then, in use, if the outer surface 95 becomes subject to fouling by barnacles, the entire cover 13′ could be reversed at least temporarily, thereby subjecting what had been the outer surface 95 to the fresh water in the space 56 and cause the barnacles to drop off.

In use, the cover 13,13′ is placed about the hull 10 so as to closely conform thereto, which squeezes some of the ambient water out through the drain 54 and over the top edge 52. The other drain tube 55 can be used to insert fresh water into the space 56 between the hull 10 and the cover 13,13′, using means known in the art. One or more pumps 57 can be positioned, for example, at a dock 58 for this purpose, or a pump onboard could be used. The fresh water remains within the space 56 as a protective “jacket.”

If barnacles have already attached to the hull 10, the fresh water jacket will cause them to detach after a period of time, since they cannot survive in fresh water. It may be beneficial to periodically flush the fresh water jacket to assist in this passive process.

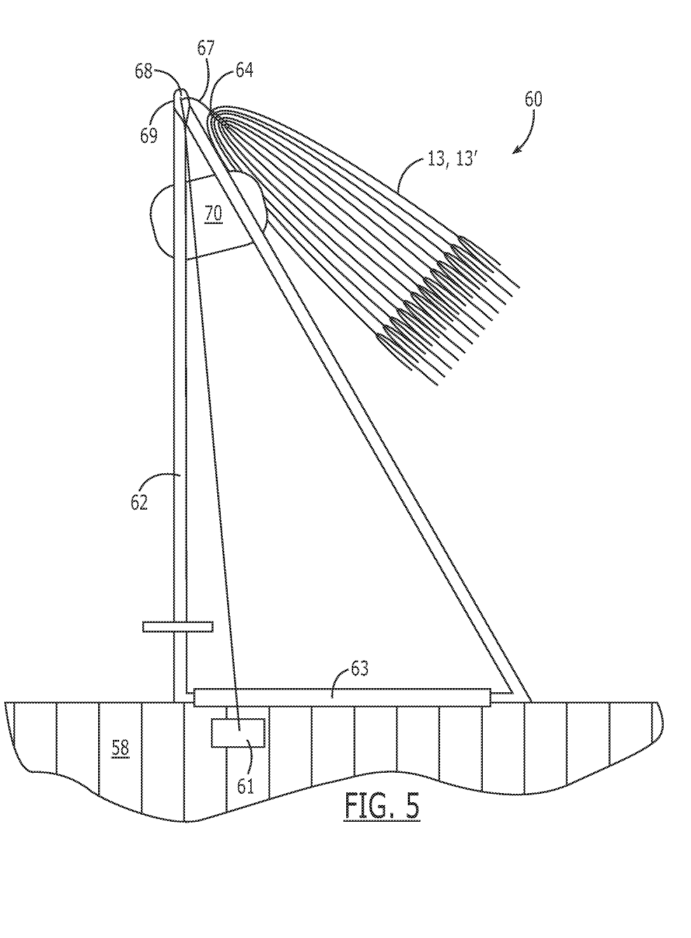

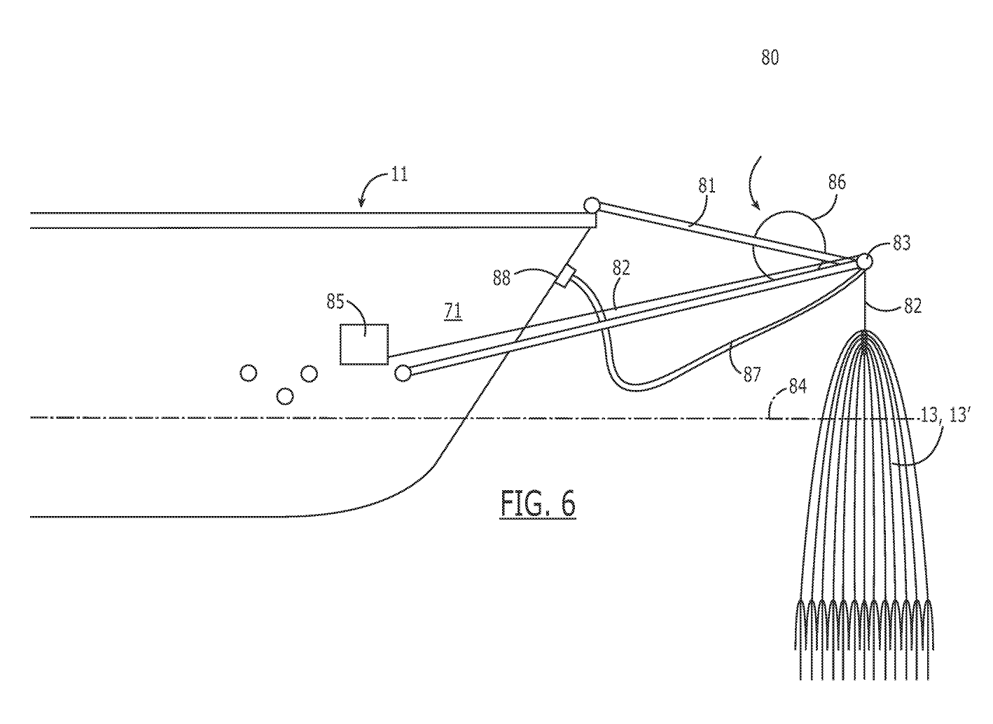

A complete system of the present invention further includes means for assisting in positioning the cover 13,13′ onto the hull 10. Two such devices are illustrated in FIGS. 5 and 6. A shore-mounted boom system 60 (FIG. 5) includes a winch 61 affixed to the dock 58 and a right-triangular support structure 62 affixed to the dock 58 with a mounting sleeve 63. The cover 13 is gathered in accordion-like fashion using a plurality of spaced-apart loops 64 affixed to either the outer surface 65 or the inner surface 46 thereof generally along a midline 66. A line 67 is threaded through the loops 64, and pulling the line 67 gathers the cover 13 to an orientation as illustrated in FIGS. 5 and 6. The line 67 passes through a guide 68 positioned adjacent a top end 69 of the support structure 62 and passes downward to the winch 61. A float 70 is also provided for maintaining the cover 13,13′ atop the water.

In use, the boom structure 62 is moved to a position where the cover 13,13′ is adjacent a bow 71 of the boat 11, and moved to let out the cover 13,13′ using the winch 61 to surround the hull 10.

In another embodiment (FIG. 6), a boat-mounted boom system 80 includes a support structure 81 that is mounted to the bow 71 of the boat 11, in similar fashion to the deck-mounted embodiment 60. Again, the cover 13,13′ is gathered in accordion-like fashion. A line 82 threaded through the loops 64 is pulled to gather the cover 13,13′. The line 82 passes through a guide 83 positioned adjacent an outer end 84 of the support structure 81 and passes inward to the winch 85. A float 86 is also provided for maintaining the cover 13,13′ atop the water. A vertical stop line 87 affixed, for example, with a cleat 88 to the hull 11 keeps the cover 13,13′ from going too far, and pulls the cover 13,13′ toward the hull 11.

In use, the boom structure 81 is moved to a position where the cover 13,13′ is adjacent the bow 71 of the boat 11, and moved to let out the cover 13,13′ using the winch 85 to surround the hull 10.

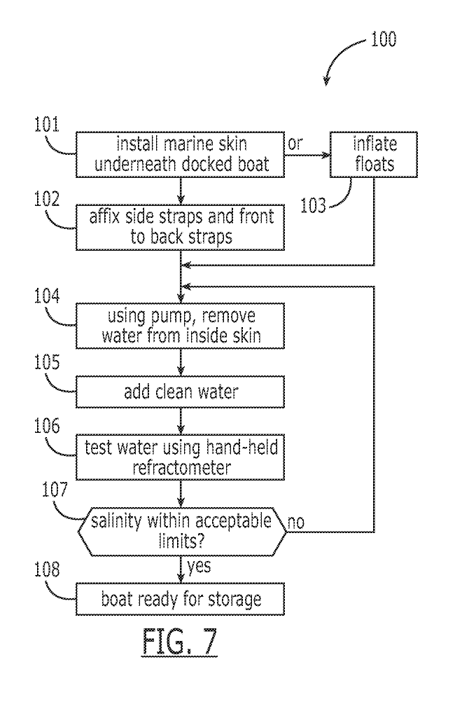

The system can further be provided with a sensor for measuring the salinity of the water in the space 56, such as a refractometer as known in the art. With this device, the system can be used (FIG. 7) with a method 100 including installing the cover 13,13′ underneath a docked boat 11 (block 101). Depending upon the embodiment used, either the tethers 48 are affixed to the hull's top edge 50 (block 102), or the flotation elements 51 are inflated (block 103). Using a pump, water is removed from inside the cover 13,13′ (block 104), and clean water is added (block 105). Periodically as desired, the refractometer is used to measure the salinity of the water (block 106). If the salinity is not within acceptable limits (block 107), the water is removed again (block 104) and replaced (block 105). If the salinity level is acceptable (block 107), the boat can be stored (block 108).

The cover 13,13′ of the present invention are intended for use when the vessel 11 is stationary, and generators or other equipment should not be used when the device 13,13′ is deployed, unless the heat-exchange channels are present, other than batteries or other shore-powered equipment. The devices 13,13′ can therefore be used when the boat 11 is in dock or anchored.

The cover 13,13′ is believed to provide numerous benefits. Hull staining or discoloration, galvanic corrosion, and encrustation from prolonged exposure to dirty, brackish, or polluted water containing fouling agents is decreased. Bottom paint to discourage marine growth is no longer needed, nor are boat lifts. The cover is easily deployed in similar fashion to applying an automobile cover, and can be performed manually or with the aid of an installation device such as a mechanical crank system. The cover 13,13′ can be easily stored in a relatively small area.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the device illustrated and described herein are by way of example, and the scope of the invention is not limited to the exact details of construction and use.

What is claimed is:

1. A protective cover for a boat hull comprising:
a substantially planar, flexible, water-impermeable cover base having substantially parallel side edges; a top edge having a notch therein, edges of the notch sealable to form a boat-hull-conforming fore portion; a bottom edge opposed to the top edge, the bottom edge having opposed outer portions substantially perpendicular to the side edges, arcuate, outwardly extending intermediate portions joining the respective outer portions at an acute angle, and a central portion substantially parallel to the outer portions joining the respective intermediate portions at an acute angle; and a rear aperture positioned laterally substantially midway between the side edges, and vertically adjacent the bottom edge outer portions;
a flexible, water-impermeable pouch adapted for placement about a bottom, water-contacting portion of a motor of a boat, top edges of the pouch affixable adjacent edges of the cover base aperture; and wherein, in use:
the edges of the notch, the bottom edge outer portions, the bottom edge intermediate portions, and the sides are sealed together to form a hull-encasing structure, the hull-encasing structure having an upper edge;
the pouch is affixed to the cover base aperture;
the hull-encasing structure and pouch are placed over the boat hull and motor;
the hull-encasing structure upper edge is retainable above a water line; and
ambient water in a space between the hull-encasing structure and pouch and the boat hull and motor is evacuated, replaced with fresh water, and permitted to remain, thereby protecting the boat hull and motor from ambient water and materials therein.

2. The protective cover recited in claim 1, further comprising, for retaining the hull-encasing structure upper edge above the water line, a plurality of spaced-apart tethers extending laterally from the cover base side edges, each tether having means affixed adjacent distal ends thereof for being removably attached to an upper edge of a hull of a boat.

3. The protective cover recited in claim 2, wherein the tethers have elasticity, and the attachment means comprise hooked elements dimensioned for extending over the hull upper edge.

4. The protective cover recited in claim 1, further comprising, for retaining the hull-encasing structure upper edge above the water line, a flotation structure affixed adjacent the hull-encasing structure upper edge.

5. The protective cover recited in claim 1, further comprising a first drain tube having a first end positioned within the space and a second, opposed end extending out from the space, the first drain tube usable for evacuating water from the space and for replacing the evacuated water with the fresh water.

6. The protective cover recited in claim 5, further comprising a second drain tube having a first end positioned within a portion of the space within the pouch and a second, opposed end extending out from the space portion, the second drain tube usable for evacuating water from the space portion and replacing the evacuated water with the fresh water.

7. The protective cover recited in claim 1, further comprising a first drain tube having a first end positioned within the space and a second, opposed end extending out from the space, the first drain tube usable for evacuating water from the space and a second drain tube having a first end positioned within the space and a second, opposed end extending out from the space, the second drain tube usable for replacing the evacuated water with the fresh water.

8. The protective cover recited in claim 1, wherein at least a portion of an inner surface of the cover base comprises a material adapted for enhancing a coefficient of friction between the cover base and the boat hull, for assisting in retaining the cover base in a desired position relative to the boat hull.

9. The protective cover recited in claim 8, wherein the material comprises a mesh material.

10. The protective cover recited in claim 9, wherein the mesh material comprises a plurality of strips thereof affixed to the cover base inner surface at least adjacent a central portion thereof in spaced relation from the side edges.

11. A system for protecting a boat hull comprising:
a hull-encasing structure having an upper edge, the hull-encasing structure shaped and dimensioned to cover a hull of a boat from a bottom thereof, so that the upper edge is above a water line of the boat, the hull-encasing structure further comprising a pouch shaped and dimensioned to encase a portion of a boat motor that in use is beneath the water line;
a pump for evacuating ambient water in a space between the hull-encasing structure and pouch and the boat hull and motor and replacing the evacuated water with fresh water, the fresh water for protecting the boat hull and motor from the ambient water and materials therein.

12. The system recited in claim 11, further comprising a first drain tube in fluid communication with the pump and having a first end positioned within the space and a second, opposed end extending out from the space, the first drain tube usable for evacuating water from the space and a second drain tube in fluid communication with the pump and having a first end positioned within the space and a second, opposed end extending out from the space, the second drain tube usable for replacing the evacuated water with the fresh water.

13. The system recited in claim 11, wherein at least a portion of the hull-encasing structure comprises a flexible, water-impervious material, and wherein the system further comprises a boom system comprising:
a boom;
a holder affixed adjacent an outer end of the boom, the holder for supporting the hull-encasing structure prior to use on the boat hull;
a line having a first end affixed to a stern end of the hull-encasing structure and a second end affixed to the boom;
means for limiting a play of the line;
wherein the boom is movable between a first position wherein the holder is in spaced relation from the boat hull and a second position wherein the holder is positioned adjacent a bow of the boat hull, the hull-encasing structure deployable onto the hull from the bow to a stern of the boat hull.

14. The system recited in claim 13, wherein the boom is affixed to one of the bow and a land-based site.

15. The system recited in claim 11, further comprising a sensor for measuring a salinity of water in the space, for providing an indication to a user that the water in the space should be replaced.
